(19) United States
(12) Patent Application Publication
(10) Pub. No.: US 2022/0062192 A1
(43) Pub. Date: Mar. 3, 2022

(54) Drug Solution And Topical Drug Delivery Device Combination

(71) Applicant: Lakeisha Perryman, New York, NY (US)

(72) Inventor: Lakeisha Perryman, New York, NY (US)

(21) Appl. No.: 17/008,805

(22) Filed: Sep. 1, 2020

(51) Int. Cl. A61K 9/70; A61K 45/06; A61K 47/38; A61K 47/32; A61K 9/00

(52) U.S. Cl. CPC A61K 9/7007; A61K 45/06; A61K 9/0014; A61K 47/32; A61K 47/38

(57) ABSTRACT

A drug solution and topical drug delivery device combination for alleviating allergy symptoms includes a sheet, which is fibrous so that the sheet is porous and absorbent. A drug solution positioned in the sheet can alter at least one biological pathway within a mammal. The sheet can be wiped on skin of the mammal so that a portion of the drug solution is delivered to the skin, wherein the drug solution is positioned to alter the biological pathway.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to drug and device combinations and more particularly pertains to a new drug and device combination for alleviating allergy symptoms.

### (2) Description of Related Art Including Information Disclosed Under 37 CFR 1.97 and 1.98

The prior art relates to drug and device combinations for topical delivery of drugs, which may comprise drug impregnated sticks, lotions, creams, gels, and the like.

### BRIEF SUMMARY OF THE INVENTION

An embodiment of the disclosure meets the needs presented above by generally comprising a sheet, which is fibrous so that the sheet is porous and absorbent. A drug solution is positioned in the sheet and is configured to alter at least one biological pathway within a mammal. The sheet is configured to be wiped on skin of the mammal so that a portion of the drug solution is delivered to the skin. The drug solution thus is configured to alter the biological pathway.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a drug solution and topical drug delivery device combination according to an embodiment of the disclosure.

FIG. 2 is a cross-sectional view of an embodiment of the disclosure.

### DETAILED DESCRIPTION OF THE INVENTION

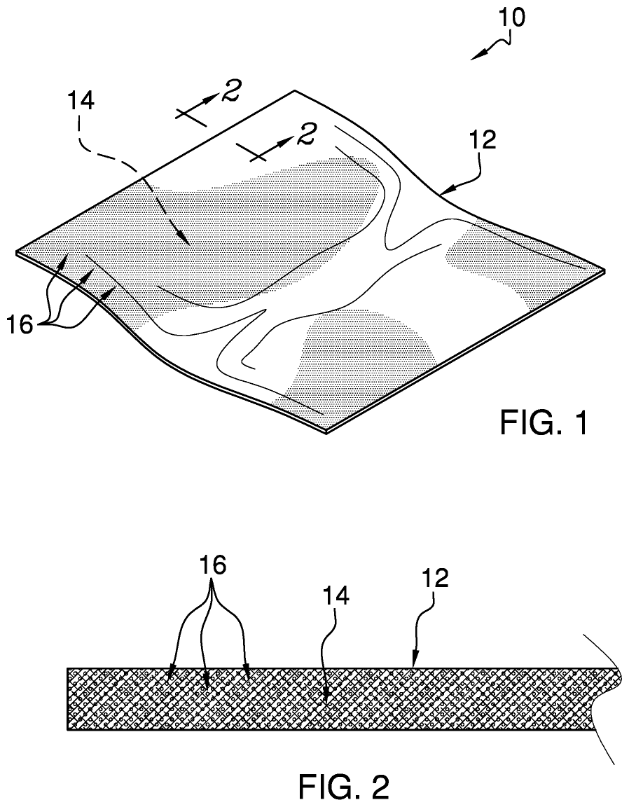

With reference now to the drawings, and in particular to FIGS. 1 and 2 thereof, a new drug and device combination embodying the principles and concepts of an embodiment of the disclosure and generally designated by the reference numeral 10 will be described.

As best illustrated in FIGS. 1 and 2, the drug solution and topical drug delivery device combination 10 generally comprises a sheet 12, which is fibrous so that the sheet 12 is porous and absorbent. A drug solution 14 is positioned in the sheet 12 and is configured to alter at least one biological pathway within a mammal. The sheet 12 is configured to be wiped on skin of the mammal so that a portion of the drug solution 14 is delivered to the skin. The drug solution 14 thus is configured to alter the biological pathway.

The sheet 12 comprises nonwoven fibers 16 so that the sheet 12 is porous and absorbent. The nonwoven fibers 16 comprise at least one of polyester, polypropylene, cellulose, and wood fiber, which should be interpreted to mean only one of polyester, polypropylene, cellulose, or wood fiber, or any combination of one or more thereof. The sheet 12 may be rectangularly shaped, as shown in FIG. 1, or alternatively shaped, such as, but not limited to, circularly shaped, ovally shaped, and the like. The sheet 12 may be individually packaged. The sheet 12 also may be one of a plurality of sheets 12, with the sheets 12 of the plurality of sheets 12 being layered so that they are sequentially dispensable from a container.

The drug solution 14 may be configured to suppress histamine production to relieve allergy symptoms resulting from allergic rhinitis, allergic conjunctivitis, atopic dermatitis, insect stings, and the like, and also to promote healing of wounds and acne.

The passage of the sheet 12 over the skin also serves to dislodge and to remove allergens from the skin.

The drug solution 14 comprises at least one of an H1-antihistamine, an H2-antihistamine, an H3-antihistamine, an H4-antihistamine, a histidine decarboxylase inhibitor, and a mast cell stabilizer, which should be interpreted to mean only one of these agents, or any combination of one or more thereof. The combination 10 enables targeted topical application of the drug solution 14 to specific areas, such as around eyes, nose, and mouth of a user, to relieve allergy symptoms.

The drug solution 14 also may be configured to alter other biological pathways, such as to achieve local anesthesia, to reduce inflammation, and the like. The drug solution 14 also may comprise an antimicrobial agent, such as an antifungal agent, antibacterial agent, and an antiviral agent.

In use, the sheet 12 is wiped over the skin to apply the drug solution 14 to targeted areas for the relief of allergy symptoms.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

1. A drug solution and topical drug delivery device combination comprising:
a sheet, the sheet being fibrous such that the sheet is porous and absorbent; and
a drug solution positioned in the sheet and being configured for altering at least one biological pathway within a mammal, wherein the sheet is configured for wiping on skin of the mammal, such that a portion of the drug solution is delivered to the skin, wherein the drug solution is configured for altering the biological pathway, wherein the drug solution comprises at least one of an H1-antihistamine, an H2-antihistamine, an H3-antihistamine, an H4-antihistamine, a histidine decarboxylase inhibitor, and a mast cell stabilizer, said drug solution further comprising an antifungal agent, an antibacterial agent, and an antiviral agent.

2. The drug solution and topical drug delivery device combination of claim 1, wherein the sheet comprises nonwoven fibers.

3. The drug solution and topical drug delivery device combination of claim 2, wherein the nonwoven fibers comprise at least one of polyester, polypropylene, cellulose, and wood fiber.

4. The drug solution and topical drug delivery device combination of claim 1, wherein the sheet is rectangularly shaped.

5. The drug solution and topical drug delivery device combination of claim 1, wherein the sheet is individually packaged.

6. The drug solution and topical drug delivery device combination of claim 1, further including the sheet being one of a plurality of sheets, the sheets of the plurality of sheets being layered such that the sheets are sequentially dispensable from a container.

7. (canceled)

8. (canceled)

9. A drug solution and topical drug delivery device combination comprising:
a sheet, the sheet being fibrous such that the sheet is porous and absorbent, the sheet comprising nonwoven fibers, the nonwoven fibers comprising at least one of polyester, polypropylene, cellulose, and wood fiber, the sheet being rectangularly shaped, the sheet being individually packaged; and
a drug solution positioned in the sheet, wherein the sheet is configured for wiping on skin of the mammal, such that a portion of the drug solution is delivered to the skin, the drug solution being configured for suppressing histamine production, the drug solution comprising at least one of an H1-antihistamine, an H2-antihistamine, an H3-antihistamine, an H4-antihistamine, a histidine decarboxylase inhibitor, and a mast cell stabilizer, said drug solution further comprising an antifungal agent, an antibacterial agent, and an antiviral agent.

10. The drug solution and topical drug delivery device combination of claim 9, further including the sheet being one of a plurality of sheets, the sheets of the plurality of sheets being layered such that the sheets are sequentially dispensable from a container.
